(19) United States
(12) Patent Application Publication
(10) Pub. No.: US 2026/0191501 A1
(43) Pub. Date: Jul. 9, 2026

(54) GUIDE SYSTEM FOR SENSOR

(71) Applicants: Texas Tech University System, Lubbock, TX (US); The Board of Trustees of the University of Alabama, Tuscaloosa, AL (US)

(72) Inventors: Jacob A. Mota, Lubbock, TX (US); Kealey J. Wohlgemuth, Lubbock, TX (US); Kelvin K. So, Tuscaloosa, AL (US); Jeffrey M. Jakalski, Tuscaloosa, AL (US); Jackson Byrd, Tuscaloosa, AL (US); Daniel R. Milligan, Tuscaloosa, AL (US)

(21) Appl. No.: 19/128,520

(22) PCT Filed: Nov. 9, 2023
(86) PCT No.: PCT/US2023/037090

§ 371 Date: May 8, 2025

Related U.S. Application Data
(60) Provisional application No. 63/423,990, filed on Nov. 9, 2022.

(51) Int. Cl. A61B 8/00; A61B 8/08

(52) U.S. Cl. CPC A61B 8/4209; A61B 8/08; A61B 8/4254

(57) ABSTRACT

The disclosed embodiments are directed to a guide system comprising a cradle configured to couple to a probe and a guide structure configured to couple to a limb of a patient, the guide structure comprising a fastening component and at least two strips configured to connect to the fastening component, wherein each of the at least two strips further comprises a guide track formed thereon, wherein the probe cradle is configured to travel along the at least two strips.

### CROSS REFERENCE TO RELATED PATENT APPLICATIONS

The present application claims the priority and benefit under 35 U.S.C. § 119(e) of U.S. Provisional Patent Application Ser. No. 63/423,990, filed Nov. 9, 2022, entitled “GUIDE SYSTEM FOR SENSOR.” U.S. Provisional Patent Application Ser. No. 63/423,990 is herein incorporated by reference in its entirety.

### TECHNICAL FIELD

The present application concerns aspects of guide systems for sensors, and in particular, guide systems for sensors to facilitate orientation of the sensor relative to a patient. Embodiments are further related to medical sensor systems. Embodiments are further related to medical equipment. Embodiments are further related to testing equipment. Embodiments further relate to a guide system for ultrasound systems used to measure muscle volume.

### BACKGROUND

Ultrasound operators have difficulty consistently achieving high quality, accurate, and repeatable scans between separate attempts and among multiple technicians.

One area where such difficulties are encountered is in scanning limbs such as legs with ultrasound sensors. Human legs can range in circumferences of 300 mm to 700 mm or even bigger ranges. The variability in limb size and shape causes variations in the orientation of the ultrasound transducer.

Likewise, operators of ultrasound devices often struggle to collect ultrasound data in a consistent way, since there is presently no means for ensuring the sensor travels along a congruent path for each pass of the ultrasound and/or between one patient and the next.

As a result, it is very difficult to accurately measure muscle volume using an ultrasound. Unfortunately, there are essentially no non-invasive methods of measuring muscle volume without the use of an ultrasound. This has resulted in an unacceptably large number of inaccurate muscle volume measurements. Therefore, there is a need to reduce user and patient variability in sensor scans, and in particular, ultrasound scans of limbs. These needs, and other needs, are at least partially satisfied by the present disclosure.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

A guide system for guiding a probe relative to a limb (or other part) of a patient or object including a probe cradle for coupling to the probe a guide structure for coupling to a patient limb is disclosed. Generally, the guide structure provides multiple tracks for guiding the probe cradle to facilitate scans of the patient. The probe cradle holds the probe or sensor and is moved along the tracks by the operator while the probe collects the information on a patient's limb. For example, the guide structure may include multiple belts wrapped around the limb at regular intervals and provides multiple tracks so that the ultrasound sensor can take predictable cross-sections of the leg at known orientations. These scans can then be stacked into a three-dimensional representation of a patient's limb.

In one aspect, the guide structure includes at least three strips, wherein each of the strips is configured to, at least partially, encircle a limb and wherein the two guide tracks are defined between a first and second one of the strips and the second and a third one of the strips.

In still another aspect, the guide structure includes a fastening component configured to maintain a spacing between the at least three strips.

In still further aspects, the flexible strips are belts, wherein the fastening component is a linear member with coupling openings and wherein the belts include posts that extend into the coupling openings.

In another aspect, each of the strips includes a fin that extends at least partially along the strip and wherein the probe cradle defines at least a pair of slots configured to extend around the fins on a pair of the strips to be guided there along.

In still further aspects, the cradle includes one of a pair of slots or a pair of fins and the guide tracks include another one of the pair of slots or fins and wherein the fins fit in the slots to facilitate orientation of the cradle with respect to the guide tracks.

In another aspect, the slots are triangular grooves.

In yet another aspect, the cradle includes a body and at least a pair of runners.

For example, the body may define a cavity configured to receive the probe. And the cavity extends through the body and the probe can access the limb through the cavity. The body may also include wings to facilitate manual gripping.

In another aspect, each of the runners includes a protrusion configured to move over the guide structure. Also, the runners may include four runners each positioned at a corner of the body and wherein a pair of the runners is configured to extend on either side of guide tracks when tracing the probe around the limb.

The present invention may also include a method of guiding a probe relative to the limb of a patient. The probe is received into a coupling relationship with a cradle. A first pair of guide tracks receives the coupled cradle and probe. The first pair of guide tracks guide the cradle at least partially around the limb to define a first pathway. A second pair of guide tracks receives the coupled cradle and probe. The second pair of guide tracks guides the cradle at least partially around the limb to define a second pathway. The pathways can have a predefined relationship with each other to facilitate assembling sensing results of the probes.

In an embodiment, a guide system comprises a cradle configured to couple to a probe and a guide structure configured to couple to a limb of a patient, the guide structure comprising a fastening component and at least two strips configured to connect to the fastening component, wherein each of the at least two strips further comprises a guide track formed thereon, wherein the probe cradle is configured to travel along the at least two strips. In an embodiment, a first end of each of the at least two strips is configured to attach to the fastening component. In an embodiment, a second end of at least one of the at least two strips is configured to attach to the fastening component. In an embodiment, the fastening component further comprises a plurality of ridges defining a plurality of coupling pads therebetween, the plurality of ridges further defining a spaced relation between the at least two strips connected to the fastening component. In an embodiment, each of the guide tracks further comprises a fin extending along a top surface of the associated strip. In an embodiment, the cradle further comprises a plurality of runners and a slot formed on each of the plurality of runners, the slot configured to engage the guide track on the at least two strips. In an embodiment, the slot comprises a triangular groove. In an embodiment, the cradle further comprises a body, the body defining a cavity configured to receive the probe. In an embodiment, the cavity extends through the body such that the sensor can access the limb of the patient through the cavity. In an embodiment, the plurality of runners include four runners each positioned at a corner of the body. In an embodiment, a first pair of the plurality of four runners is configured to engage one of the at least two strips, and wherein a second pair of the plurality of four runners is configured to engage the other of the at least two strips.

In another embodiment, a system comprises a cradle configured to couple to a probe, the probe further comprising, a body defining a cavity configured to receive the probe; and a plurality of runners; and a guide structure, the guide structure comprising a plurality of strips, wherein each of the plurality of strips further comprises a guide track formed thereon and a fastening component comprising a plurality of coupling pads, the plurality of coupling pads defining a spaced relation between each of the plurality of strips connected to the fastening component. In an embodiment, the plurality of runners include four runners each positioned at a corner of the body and configured to engage the guide track. In an embodiment, the fastening component further comprises a stiffened strip body and a hook and loop fastener at each of the plurality of coupling pads. In an embodiment, each of plurality of strips comprises a hook and loop fastener on a first end of the strip. In an embodiment, at least one of the plurality of strips further comprises a hook and loop fastener on a second end of the strip. In an embodiment, each of the plurality of strips further comprise an arced body. In an embodiment, at least one of the arced bodies of the plurality of strips is a different length than at least one other of the arced bodies of the plurality of strips.

In an embodiment, a system comprises a cradle configured to couple to a probe, the probe further comprising: a body defining a cavity configured to receive the probe and a plurality of runners; and a guide structure, the guide structure comprising: a plurality of flexible strips, wherein each of the plurality of flexible strips further comprise a guide track formed thereon and a fastening component comprising a plurality of coupling pads, the plurality of coupling pads defining a spaced relation between each of the plurality of strips connected to the fastening component. In an embodiment, each of the plurality of flexible strips comprises thermoplastic polyurethane.

The foregoing and other features and advantages of the disclosure will become more apparent from the following detailed description, which proceeds with reference to the accompanying figures.

### BRIEF DESCRIPTION OF THE FIGURES

The accompanying figures, in which like reference numerals refer to identical or functionally similar elements throughout the separate views and which are incorporated in and form a part of the specification, further illustrate the embodiments and, together with the detailed description, serve to explain the embodiments disclosed herein.

FIG. 1 depicts aspects of a guide system, in accordance with the disclosed embodiments;

FIG. 2 depicts a fastening component associated with a guide system, in accordance with the disclosed embodiments;

FIG. 3 depicts aspects of a cradle associated with a guide system, in accordance with the disclosed embodiments;

FIG. 4A depicts a strip associated with a guide system, in accordance with the disclosed embodiments;

FIG. 4B depicts another embodiment of a strip associated with a guide system, in accordance with the disclosed embodiments;

FIG. 5 depicts another embodiment of a guide system, in accordance with the disclosed embodiments;

FIG. 6 depicts aspects of a guide system, in accordance with the disclosed embodiments;

FIG. 7 depicts aspects of another cradle associated with a guide system, in accordance with the disclosed embodiments;

FIG. 8 depicts aspects of an embodiment of a guide system, in accordance with the disclosed embodiments;

FIG. 9 depicts aspects of an embodiment of a fastening component associated with a guide system, in accordance with the disclosed embodiments;

FIG. 10 depicts aspects of an embodiment of a strip associated with a guide system, in accordance with the disclosed embodiments; and

FIG. 11 depicts steps associated with a method for using the disclosed guide system, in accordance with the disclosed embodiments.

### DETAILED DESCRIPTION

Embodiments and aspects of the disclosed technology are presented herein. The particular embodiments and configurations discussed in the following non-limiting examples can be varied, and are provided to illustrate one or more embodiments, and are not intended to limit the scope thereof.

Reference to the accompanying drawings, in which illustrative embodiments are shown, are provided herein. The embodiments disclosed can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

The present disclosure can be understood more readily by reference to the following detailed description, examples, drawings, and claims, and their previous and following description. However, before the present articles, systems, and/or methods are disclosed and described, it is to be understood that this disclosure is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the disclosure is provided as an enabling teaching of the disclosure in its best, currently known aspect. To this end, those skilled in the relevant art will recognize and appreciate that many changes can be made to the various aspects of the disclosure described herein while still obtaining the beneficial results of the present disclosure. It will also be apparent that some of the desired benefits of the present disclosure can be obtained by selecting some of the features of the present disclosure without utilizing other features. Accordingly, those of ordinary skill in the pertinent art will recognize that many modifications and adaptations to the present disclosure are possible and may even be desirable in certain circumstances and are a part of the present disclosure. Thus, the following description is again provided as illustrative of the principles of the present disclosure and not in limitation thereof.

Further, the terms “coupled” and “associated” generally mean electrically, electromagnetically, and/or physically (e.g., mechanically or chemically) coupled or linked and do not exclude the presence of intermediate elements between the coupled or associated items.

The present invention may be understood more readily by reference to the following detailed description of various aspects of the invention and the examples included therein and to the figures and their previous and following description.

The disclosed embodiments are generally directed to a guide system. In general, the guide system 10 includes a cradle 14 configured to couple to a probe 12, and a guide structure 16 generally configured to couple to a limb of a patient, the guide structure comprising a fastening component and strips configured to connect to the fastening component. Each of the at least two strips further comprises a guide track formed thereon, and the probe cradle is configured to travel along the at least two strips. The first end of each of the strips is configured to attach to the fastening component. In certain embodiment, the second end of each of the strips can also be configured to attach to the fastening component.

The fastening component generally comprises a plurality of ridges defining a plurality of coupling pads therebetween, the plurality of ridges further defining a spaced relation between the at least two strips connected to the fastening component. Each of the guide track can further comprise a fin extending along a top surface of the associated strip. In certain embodiment, the cradle further comprises a plurality of runners and a slot formed on each of the plurality of runners, the slot configured to engage the guide track on the strips.

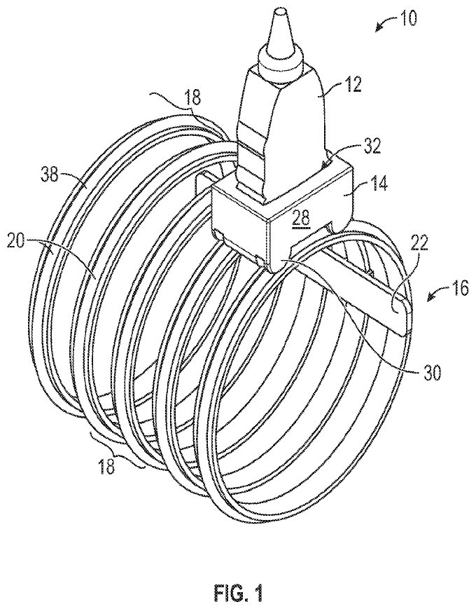

FIG. 1 illustrates an exemplary guide system 10 for guiding a probe 12 relative to a limb of a patient. The guide system 10 generally includes a cradle 14 for coupling to the probe 12 and a guide structure 16 for coupling to the limb of the patient. The guide system 10, for example, can include guide tracks 18 having a (relatively) fixed relationship to the limb of the patient and between the guide tracks themselves.

When the probe cradle 14 is coupled to the probe 12 and the probe cradle 14 is moved along the guide tracks 18, the probe 12 can be used to collect one or more scans of an anatomical feature of the patient. In certain embodiments, the anatomical feature can be a leg, arm, or other body part. The scans each have a predefined relationship with each other, facilitated by the guide system 10, that advantageously restrains deviation of rotational angles to within about 5% of a control angle. In an exemplary embodiment, the array of scans provide a series of laterally adjacent scans that can be used in measuring, for example, tissue volume of a section of a body part.

Although the probe 12 illustrated herein is an ultrasound sensor or probe, the guide system 10 can be employed with a range of sensors that benefit from maintenance of known pathways relative to each other and/or a patient, especially a patient limb or other appendage. The term “limb” as used herein, is not strictly indicative of arms or legs and can include any portion of any construct that has relatively mobile or compressible (and therefore changing) geometry that would introduce variability in a scan pathway.

The terms “track” or “tracks” herein refer to pairs of structure between which is defined some space, and along which structure the probe cradle 14 can be supported and guided. Preferably the sensing element of the probe 12 is narrower than the tracks 18 so as to avoid interference of the strips defining the guide tracks 18 in the sensing function.

The terms “strip” or “strips” refers to any linear members that can be configured to, at least partially, encircle a limb and define therebetween tracks. Strips for example can be comprised of flexible strips such as belts that can encircle a limb of varying cross-sectional shape.

The guide structure 16 can include a plurality of strips 20 or other members between pairs of which are defined the tracks 18, as well as additional structure configured to fasten and maintain the relationship between the strips 20. In certain embodiments, the strips 20 can be flexible. In other embodiments, the strips can be ridged or semi-rigid. The strips can comprise thermoplastic polyurethane.

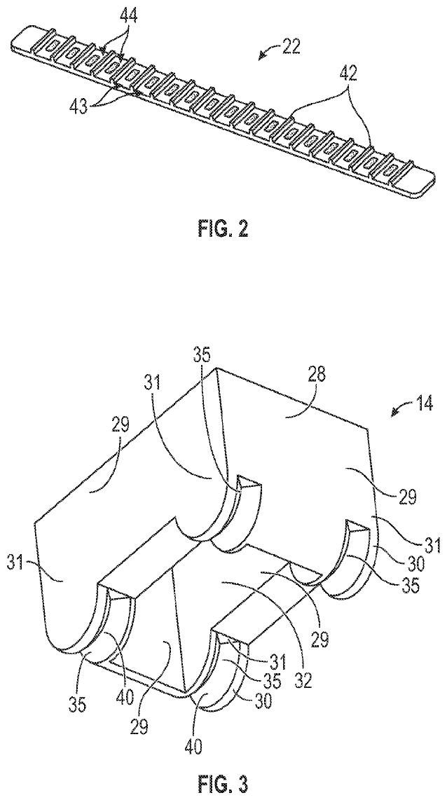

The guide structure 16 can further include a fastening component 22. The fastening component 22 may be a linear member. FIG. 2 illustrates aspects of one embodiment of a fastening component 22. As illustrated, the fastening component includes a plurality of ridges 42 defining a plurality of coupling pads 43 therebetween. The plurality of ridges 42 further define a spaced relation between the strips 20 connected to the fastening component 22. In certain embodiments fasteners 44 are disposed on the coupling pads 43. The fasteners 44 can comprise hook and loop fasteners, or other such, non-permanent fastening means.

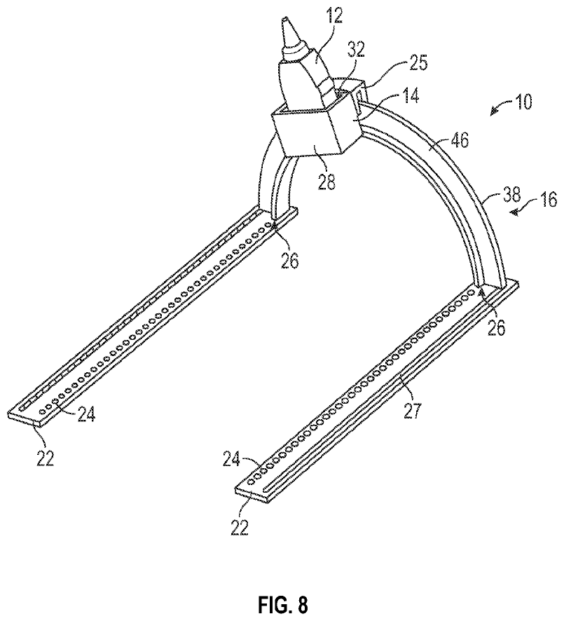

In other embodiments, as shown in FIG. 8, the fastening component 22 defines a plurality of openings 24. The flexible strips 20 may include posts 26 configured to insert into or otherwise mate with the openings 24. In this manner, the fastening component 22 can help maintain the spaced relationship between the flexible strips 20. The strips 20 may include additional structure such as fins 38 to guide the probe cradle 14 there along.

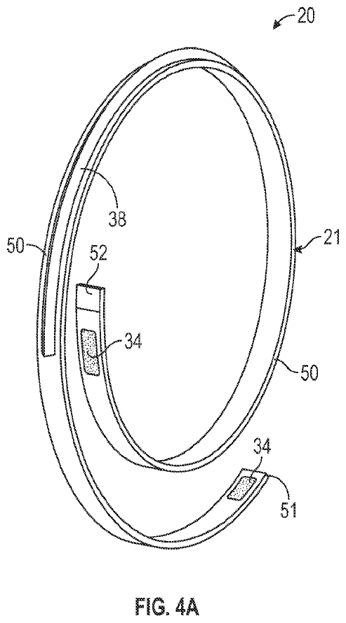

FIG. 3 illustrates aspects of a cradle 14 in accordance with the disclosed embodiments. In this exemplary embodiment, the cradle 14 includes a body 28 and a set of runners 30. The cradle defines a cavity 32 configured to accept the sensor 12. The body 28 can have a generally rectangular structure with four perpendicular sidewalls 29 that surround the cavity 32. The cavity 32 is configured to have a shape that can fit (such as a press fit) with a sensor end of the probe 12, as shown in FIG. 1.

For example, the body 28 can define a cavity 32 having a rectangular shape configured to fit the rectangular shape of the head of the probe 12 (e.g., an ultrasound probe), as shown. Generally, the cavity 32 passes through the body and is open so that the probe 12 has unblocked line of site to the patient's body part as the probe 12 and cradle 14 circumnavigate the patient's body part along the guide tracks 18. Preferably components of the body 28 and the remaining parts of the guide structure 16 are configured for patient comfort.

Although the illustrated body 28 of the cradle 14 and the cavity 32 defined within are rectangular in shape in the exemplary illustration of FIG. 3, the body 28 and cavity 32 can be configured in a range of shapes and are generally shaped to receive and maintain a position and orientation of the probe 12. Generally, the body 28 therefore benefits from being sufficiently large to define the cavity 32 so as to securely couple with the probe 12. The size, shape and number of probes may vary for various applications. As such, the size and shape of the cavity 32 and the surrounding body 28 may be adapted thereto. The probe 12 for example may have a cylindrical shape and thus the body 28 and the cavity 32 of the cradle 14 can also have a cylindrical shape. Additionally, the probe 12 may have an irregular shape and the cavity 32 need not have the same shape as the body-instead the cavity may have some congruencies that facilitate the fit and stability of the coupling.

The body 28 of the cradle 14 can also be shaped to provide additional or improved gripping surfaces for manual movement and manipulation of the combined coupled cradle 14 and probe 12. The body 28 having additional bulk, for example, provides for an improved grip. The body 28 may also include gripping features such as wings, rings, or pistol grip shapes. Additionally, the cradle 14 may be shaped to fit or couple with a motorized driver that can urge the cradle 14 and probe 12 along the guide structure 16. The coupling between the body 28 of the cradle 14 can also be designed so that the probe 12 is not fixed in position relative to the body 28. Instead, the coupling can allow the probe 12 to move within a tolerance within the body 28 as it circumnavigates a patient's body part.

As shown in FIGS. 1 and 3, the runners 30 can be positioned at the four corners 31 of the body 28 and extend away from the body 28 and the face of the probe 12. The runners can have half-round bodies 32 and raised, semispherical protrusions extending from the half-round shapes. The shapes of the runners 30, and their positioning at the four corners 31 of the body 28, are selected for easy sliding and stabilization of the position of the cradle 14 relative to the tracks 18 of the guide structure 16. Four points of contact at four corners 31 of the body 28 helps to control, not just the position of the probe 12, but also its orientation in three-dimensional space. The runners 30 include slots 35 configured to engage the guide tracks 18 on the strips 20. In certain embodiments, the slots 35 are configured as a triangular groove configured to match the profile of the fins 38 on the tracks guide tracks 18.

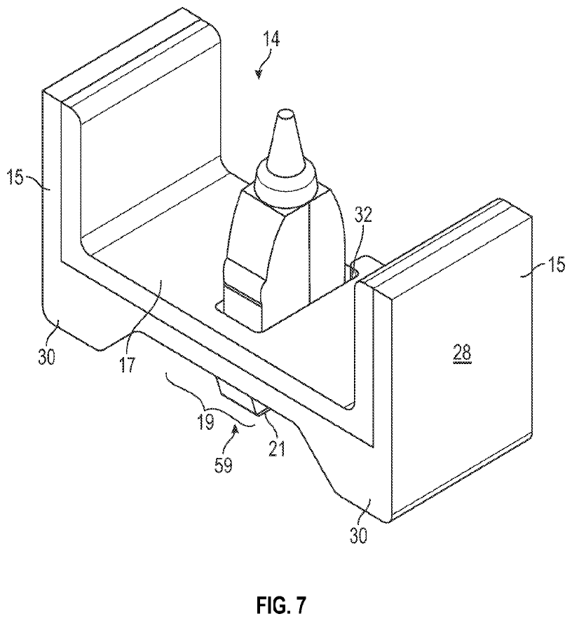

FIG. 7 shows another configuration of a cradle 14 for the ultrasound probe 12. For example, the cradle 14 has a body 28 with a U-shaped configuration, wherein the body 28 includes two arms 15. In this example, the two arms 15 of the “U” extend in a direction opposite the sensor end 59 of the probe 12 and provide gripping features for the cradle 14. In such an example, the U-shaped body 28 includes a base 17 disposed between the two arms 15, wherein the base 17 has a central portion 19. In some examples, the central portion 19 of the base 17 of the “U” defines a cavity 32 which, as in FIG. 2, has a rectangular shape to fit the end of the rectangular probe 12. The U-shape may be formed of two separate pieces that fit together to entrap the probe 12. In FIG. 7, the cradle 14 includes a pair of runners 30 protruding outward from the body 28 extending in a parallel arrangement in the same direction as the sensor end 59 of the probe 12. The runners 30 are tapered protrusions that present parallel rectangular surfaces for engagement with the guide structure 16. The runners can include grooves with, for example, a triangular cross-section (4.9 mm sides and a 6.5 mm opening) to receive a congruent triangular ridge on the flexible strips 20, as described more below. The probe cradle for sonogram applications can have a width, for example, of 41.5 mm to 70 mm. The length of the cradle can be 63 mm to 65 mm.

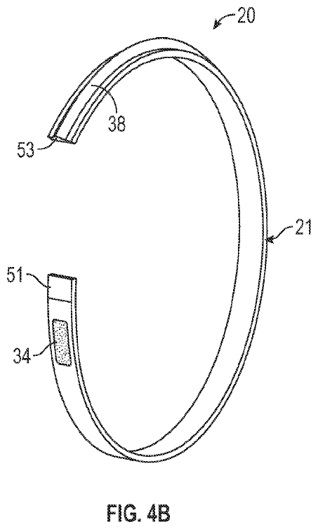

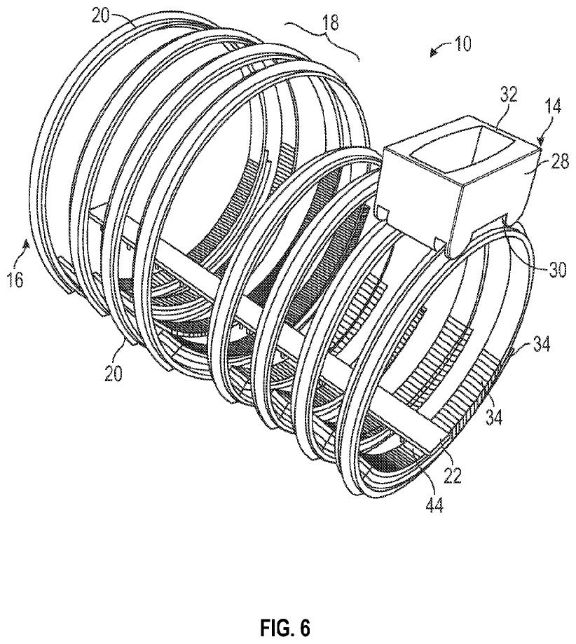

FIG. 4A illustrates aspects of the strips 20 in accordance with the disclosed embodiments. In certain embodiments, the strips 20 may be comprised of a flexible strip-shaped material that has an elongated and arced body structure 21 with parallel edges 50, a first end 51, and a second end 52. In this embodiment, the strips 20 generally can have sufficient length and flexibility to encircle and conform to the patient's limb. The strips 20 may also include hook and loop surfaces 34 on first end 51 and/or second end 52, that facilitate closing the loop of the strips snugly about the patient's limb by connecting to each other, or to hook and loop fasteners 34 on the fastening component 22. The hook and loop surfaces 34, for example, may be hook and loop tape on the inside of the strips as shown in FIG. 6. These hook and loop surfaces can also interact with complementary hook and loop surfaces 34 on the fastening component 22 for example. The strips may have a width of about 12.7 mm to 20 mm for a quadriceps application, although in other applications other widths can be used. The strip 20 further includes a fin 38. The fin 38 is configured to couple with or otherwise engage the slots 35 of the cradle 14. In certain embodiments, the fins 38 have a triangular profile.

FIG. 4B illustrates an exemplary strip 20, with a shortened length, in accordance with the disclosed embodiments, in this embodiment the strip 20 can be formed with a stiffer material, that is sufficiently rigid to generally hold its arced shape, without being so rigid that it does not generally conform to the body part it is placed around. The first end 51 of the strip 20 includes hook and loop fastener 34. However, the second end 53 is shortened so that the strip 20 does not create a full curl loop. Likewise, the second end 53 does not include a hook and loop fastener. The strip 20 thus comprises an arced body 21. The strip 20 can further include a fin 38. The fin 38 is configured to couple with or otherwise engage the slots 35 of the cradle 14. In certain embodiments, the fins 38 have a triangular profile.

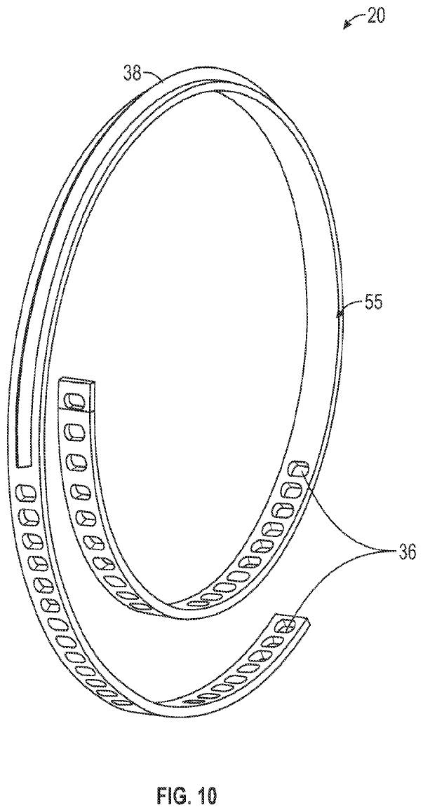

FIG. 10 shows another version of the strips 20 wherein each strip is a belt 55 with a plurality of belt openings 36 that are spaced at intervals along end portions of the strips 20. In this manner, the openings at opposite ends of the strip 20 can be overlapped in registration with each other to receive a fastener therethrough. This is yet another way to secure the strips about a patient's limb with varying shape and/or diameter.

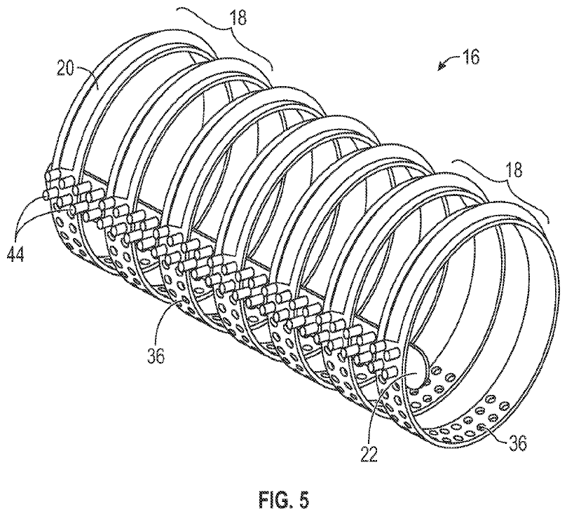

The strips 20 can also include structure for interacting with the cradle 14, such as ridges or fins 38 that extend away from the patient's body part, as shown in FIGS. 5, 8 and 10. These ridges or fins 38, for example, can extend into notches or slots 35 (shown in FIG. 2) in the runners 30 of the cradle 14. The structure can also be reversed with the ridges, fins or other structure extending from the cradle 14 and into notches in the strips or other portions of the guide structure 16. The ridges or fins 38 may have a range of shapes, such as triangular or rectangular and a range of heights and thicknesses, with the objective being smooth, stable guidance but not so much structure as to interfere with switching tracks 18. Generally, a ridge is shorter and thicker than a fin and a notch is shallower than a slot. Slots are more congruent to fins and such a coupling provides more stability but may slow the process of uncoupling. Aspects of the present invention include different amounts of congruency and tightness of fit to adjust to the desired stability and expected experience of the operator.

FIG. 2 shows one type of fastening component 22 that includes a strip with a ladder of regularly spaced ridges 42 extending along its length. As shown in FIG. 1, these ridges can space apart the strips 20 extending around the patient's limb. The ridges 42 are spaced apart at desired intervals such that they space the corresponding flexible strips 20 apart to define the guide tracks 18 for controlled operation of the coupled probe 12 and cradle 14.

The fastening component 22 can have other structure that facilitates the spacing and interconnection of the strips to form the guide structure 16 and define the guide tracks 18. For example, as shown in FIG. 5, the fastening component 22 may have a strip shape but with pairs of posts 44 that extend along the strip at regular intervals. The flexibles strips 20 further define belt openings 36 that, when overlapped around a body part and then transfixed by the posts 44, maintain the flexible strips 20 in a spaced arrangement. The spacings between adjacent flexible strips 20 form the guide tracks 18. The belt openings 36 for example may be 5 mm and the prongs or posts 44 may be 4.8 mm in diameter. The posts 44 can be about 8 mm high in certain embodiments. In other cases, the posts can be of other heights. It should be appreciated that these dimension are exemplary and other sizes can also be sued.

In another variation, the fastening component 22 can include hook and loop structure 34, as shown in FIG. 6, that attaches to complementary hook and loop structure 34 on the flexible strips 20. An advantage of the hook and loop connection is increased flexibility in the placement of the flexible strips 20. The hook and loop connection can be combined with the ridges 42 for predictable spacing but secure and easy rearrangement. The fastening component 22 may be about 20 mm wide and 310 mm long.

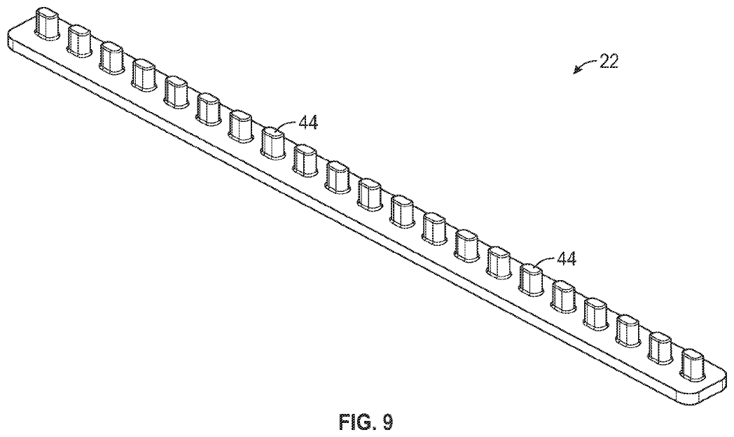

FIG. 9 shows yet another variation in the fastening component 22 which includes a strip structure with single pegs or posts 44 each having a rounded rectangular cross section.

It should be noted that guide systems 10 may have a range of varied structural features as long as these structures interact to define pathways for moving sensors or probes 12 relative to patient limbs in known and/or predictable ways that facilitate an understanding of the information collected from the probes. The desired outcome would include facilitating the taking of equally spaced, repeatable scans and a universal fit to the limb (such as quadricep muscle) of any patient. Use of versions including an adjustable belt or strip, fastening plate and probe cradle has the advantage of adjustability as the limb changes size along its length, while still facilitating taking regular, equally spaced scans with minimal transverse movement of the probe.

FIG. 8, for example, shows another guide system 10 of the present invention. This version includes a series of one or more shaped arches 46 that extend in a spaced arrangement along the limb of the patient. The arches 46 define a radius. The arches 46 are interconnected by a pair of parallel fastening components 22 which can be 400 mm in length. The parallel fastening components 22 have a series of openings 24 that facilitate connection with complementary structure (pegs-not shown) on the ends of the arches 46. The parallel fastening components 22 may also define guide holes 27 that receive complementary structure on the ends of the arches, wherein the guide holes 27 further limit movement of the arches 46 relative to the fastening components 22. Each of the arches 46 includes a fin 38 upon which a slot 25 defined on the side of a cradle 14 rides. Like the other cradles shown herein, the cradle 14 includes a body 28 that defines a cavity 32 configured to receive the sensing end 19 of the probe 12. Generally, the arches 46 may be comprised of a more rigid material and the height of the fins 38 and depth of the slot 25 in the side of the cradle 14 can improve accuracy and repeatability of the sensor 12 and cradle 14 coupling relative to the patient's leg.

However, use of flexible strips 20 facilitate adjustability to conform to the shape and size of the patient's leg. FIG. 6, for example, shows how the flexible strips 20 use hook and loop fasteners 34 for adjustment to different diameters of the strips 20, for different parts of a patient limb-such as an upper leg (above the knee, over the quadriceps) and lower leg (below the knee, over the calf).

The portions of the guide structure 16 preferably can withstand forces of up to 10 Newtons and deflect less than 1 mm for improved accuracy of tracking. (Such a force is much higher than expected in a normal scan.) Advantages of the guide system 10 include ease of use and cleaning, inexpensive construction (such as via 3D printing), wearer comfort, interchangeable parts, ability to scan limbs of varying sizes such as quadriceps varying in circumference (e.g., from 300 mm to 700 mm), deviations from control of less than 5% and even less than 3%. Deviations of less than 5% correlate with maintaining scan quality.

The guide system 10 can be used in association with real-time ultrasound sensors such as a L4-12t General Electric Ultrasound Probe, used to scan a patient body part (e.g., quadriceps) and create sonogram images. Ultrasound gel can be used to reduce the air between the patient's skin and the transducer. Multiple scans can be taken before and after resistance training and the scans can be pieced together for a three-dimensional image of the patient's quadriceps. The ease of cleaning the system is facilitated through use of ultrasound gel.

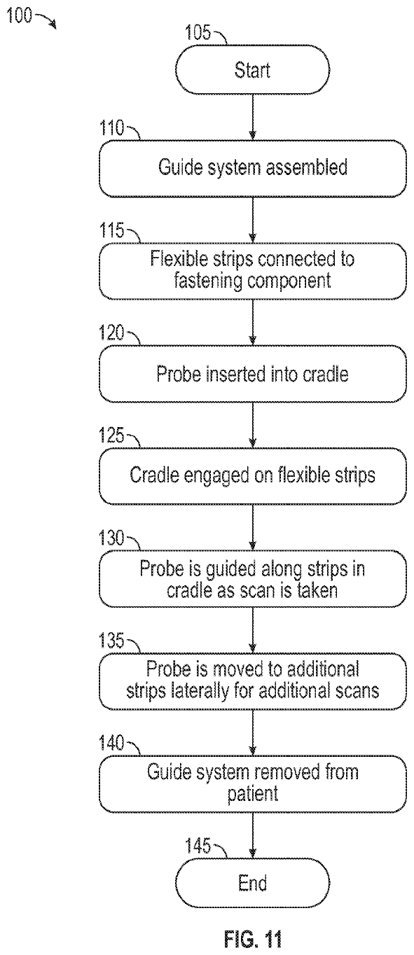

FIG. 11 illustrates steps associated with a method 100 for measuring tissue with the guide system 10 as detailed herein. The method starts at step 105.

At step 110 the guide system 10 can be assembled. In certain embodiments, this can include installing the guide system 10 on the patient. For example, in the case of a leg scan, the patient can lie on their back on a table with their knee bent, effectively forming a triangle with the table. The fastening component 22 can be placed along the patient's hamstring, with the ridges 42 facing away from the skin.

Next, at step 115, one or more of the flexible strips or belts 20 can be joined to the fastening component 22. For example, the hook and loop on the end of the strip 20 is connected to the complementary hook and loop features on the fastening component 22. Or, in the case of embodiments making use of the ridges 42 or posts 44, the openings 24 in the fastening component 22 can be pushed over posts 26 on the belt 20. The strip 20 can then be fit snugly around the leg, without tightly squeezing or constraining the leg muscles. In certain embodiments, the second end of belt can be similarly attached to the other end to the fastening component 22, in the same manner as the first end. In other embodiments, where the second end is shortened, the second end naturally extends around the leg without additional connection. This process can be repeated for each flexible strip 20 necessary for collecting the desired number of readings. In many cases this might be 7-8 strips although the number will ultimately depend on the anatomy of the patient and the specific application.

At step 120, the probe can be inserted into the cradle and at step 125, the cradle can be engaged to the flexible strips 20. The guide system 10 is now ready for use.

At step 130, the probe is guided along the exterior circumference of the patient's leg as a scan is taken. The guide system 10, allows the probe to be moved evenly along the circumference. After the first scan is complete, the probe and cradle are moved laterally to the next flexible strip 20, so that another scan can be taken, as shown at step 135. This process is repeated for the necessary number of scans to collect the desired data.

Once scanning is complete, the guide system 10 can be removed from the patient at step 140 and the method ends at step 145.

The disclosed systems and methods provide a means for easily completing a scan even for technicians of varying skill levels. Angular data can be collected (roll, pitch, and yaw) as the probe is translated across the body part. The standard deviation for roll, pitch and yaw has been determined for a variety of technicians with the level of deviation being very low. Indeed, the guide system 10 disclosed herein has been shown to reduce error to under 5% even for new technicians, greatly improving the accuracy and fidelity of the associated measurements.

In view of the many possible aspects to which the principles of the disclosed disclosure can be applied, it should be recognized that the illustrated aspects are only some examples of the disclosure and should not be taken as limiting the scope of the disclosure. Rather, the scope of the disclosure is defined by the following claims. We, therefore, claim as our disclosure all that comes within the scope and spirit of these claims.

Based on the foregoing, it can be appreciated that a number of embodiments, preferred and alternative, are disclosed herein. In an embodiment, a guide system comprises a cradle configured to couple to a probe and a guide structure configured to couple to a limb of a patient, the guide structure comprising a fastening component and at least two strips configured to connect to the fastening component, wherein each of the at least two strips further comprises a guide track formed thereon, wherein the probe cradle is configured to travel along the at least two strips.

In an embodiment a first end of each of the at least two strips is configured to attach to the fastening component. In an embodiment, a second end of at least one of the at least two strips is configured to attach to the fastening component.

In an embodiment, the fastening component further comprises a plurality of ridges defining a plurality of coupling pads therebetween, the plurality of ridges further defining a spaced relation between the at least two strips connected to the fastening component. In an embodiment, each of the guide tracks further comprises a fin extending along a top surface of the associated strip.

In an embodiment the cradle further comprises a plurality of runners and a slot formed on each of the plurality of runners, the slot configured to engage the guide track on the at least two strips. In an embodiment, the slot comprises a triangular groove. In an embodiment, the cradle further comprises a body, the body defining a cavity configured to receive the probe. In an embodiment, the cavity extends through the body such that the sensor can access the limb of the patient through the cavity. In an embodiment, the plurality of runners include four runners each positioned at a corner of the body. In an embodiment, a first pair of the plurality of four runners is configured to engage one of the at least two strips, and wherein a second pair of the plurality of four runners is configured to engage the other of the at least two strips.

In another embodiment a system comprises a cradle configured to couple to a probe, the probe further comprising, a body defining a cavity configured to receive the probe; and a plurality of runners; and a guide structure, the guide structure comprising a plurality of strips, wherein each of the plurality of strips further comprises a guide track formed thereon and a fastening component comprising a plurality of coupling pads, the plurality of coupling pads defining a spaced relation between each of the plurality of strips connected to the fastening component. In an embodiment, the plurality of runners include four runners each positioned at a corner of the body and configured to engage the guide track.

In an embodiment, the fastening component further comprises a stiffened strip body and a hook and loop fastener at each of the plurality of coupling pads.

In an embodiment, each of plurality of strips comprises a hook and loop fastener on a first end of the strip. In an embodiment, at least one of the plurality of strips further comprises a hook and loop fastener on a second end of the strip. In an embodiment, each of the plurality of strips further comprise an arced body. In an embodiment, at least one of the arced bodies of the plurality of strips is a different length than at least one other of the arced bodies of the plurality of strips.

In an embodiment, a system comprises a cradle configured to couple to a probe, the probe further comprising: a body defining a cavity configured to receive the probe and a plurality of runners; and a guide structure, the guide structure comprising: a plurality of flexible strips, wherein each of the plurality of flexible strips further comprise a guide track formed thereon and a fastening component comprising a plurality of coupling pads, the plurality of coupling pads defining a spaced relation between each of the plurality of strips connected to the fastening component. In an embodiment, each of the plurality of flexible strips comprises thermoplastic polyurethane.

It will be appreciated that variations of the above-disclosed and other features and functions, or alternatives thereof, may be desirably combined into many other different systems or applications. Also, it should be appreciated that various presently unforeseen or unanticipated alternatives, modifications, variations, or improvements therein may be subsequently made by those skilled in the art which are also intended to be encompassed by the following claims.

What is claimed is:

1. A guide system comprising:
a cradle configured to couple to a probe; and
a guide structure configured to couple to a limb of a patient, the guide structure comprising:
a fastening component; and
at least two strips configured to connect to the fastening component, wherein each of the at least two strips further comprises a guide track formed thereon, wherein the probe cradle is configured to travel along the at least two strips.

2. The guide system of claim 1, wherein a first end of each of the at least two strips is configured to attach to the fastening component.

3. The guide system of claim 2 wherein a second end of at least one of the at least two strips is configured to attach to the fastening component.

4. The guide system of claim 1, wherein the fastening component further comprises:
a plurality of ridges defining a plurality of coupling pads therebetween, the plurality of ridges further defining a spaced relation between the at least two strips connected to the fastening component.

5. The guide system of claim 1, wherein each of the guide tracks further comprises a fin extending along a top surface of the associated strip.

6. The guide system of claim 1, wherein the cradle further comprises:
a plurality of runners; and
a slot formed on each of the plurality of runners, the slot configured to engage the guide track on the at least two strips.

7. The guide system of claim 6, wherein the slot comprises a triangular groove.

8. The guide system of claim 1, wherein the cradle further comprises:
a body, the body defining a cavity configured to receive the probe.

9. The guide system of claim 8, wherein the cavity extends through the body such that the sensor can access the limb of the patient through the cavity.

10. The guide system of claim 9, wherein the plurality of runners include four runners each positioned at a corner of the body.

11. The guide system of claim 10 wherein a first pair of the plurality of four runners is configured to engage one of the at least two strips, and wherein a second pair of the plurality of four runners is configured to engage the other of the at least two strips

12. A system comprising:
a cradle configured to couple to a probe, the probe further comprising:
a body defining a cavity configured to receive the probe; and
a plurality of runners; and
a guide structure, the guide structure comprising:
a plurality of strips, wherein each of the plurality of strips further comprises a guide track formed thereon; and
a fastening component comprising a plurality of coupling pads, the plurality of coupling pads defining a spaced relation between each of the plurality of strips connected to the fastening component.

13. The system of claim 12, wherein the plurality of runners include four runners each positioned at a corner of the body and configured to engage the guide track.

14. The system of claim 12, wherein the fastening component further comprises:
a stiffened strip body; and
a hook and loop fastener at each of the plurality of coupling pads.

15. The system of claim 14 wherein each of plurality of strips comprises:
a hook and loop fastener on a first end of the strip.

16. The system of claim 15 wherein at least one of the plurality of strips further comprises:
a hook and loop fastener on a second end of the strip.

17. The system of claim 12 wherein each of the plurality of strips further comprise:
an arced body.

18. The system of claim 18 wherein at least one of the arced bodies of the plurality of strips is a different length than at least one other of the arced bodies of the plurality of strips.

19. A system comprising:
a cradle configured to couple to a probe, the probe further comprising:
a body defining a cavity configured to receive the probe; and
a plurality of runners; and
a guide structure, the guide structure comprising:
a plurality of flexible strips, wherein each of the plurality of flexible strips further comprise a guide track formed thereon; and
a fastening component comprising a plurality of coupling pads, the plurality of coupling pads defining a spaced relation between each of the plurality of strips connected to the fastening component.

20. The system of claim 19, wherein each of the plurality of flexible strips comprises thermoplastic polyurethane.
